(19) United States
(12) Patent Application Publication
(10) Pub. No.: US 2008/0065025 A1
(43) Pub. Date: Mar. 13, 2008

(54) SAFETY NEEDLE ASSEMBLY

(75) Inventors: RICHARD P. JENKINS, BLUFFDALE, UT (US); S. RAY ISAACSON, ROY, UT (US)

Correspondence Address: DAVID W. HIGHET, VP AND CHIEF IP COUNSEL;BECTON, DICKINSON AND COMPANY, 1 BECTON DRIVE, MC 110, FRANKLIN LAKES, NJ, 07417-1880, US

(73) Assignee: BECTON DICKINSON AND COMPANY, FRANKLIN LAKES, NJ (US)

(21) Appl. No.: 11/530,975

(22) Filed: Sep. 12, 2006

(51) Int. Cl. A61M 5/32

(52) U.S. Cl. 604/192

(57) ABSTRACT

Embodiments of the invention pertain to safety needle assemblies. In one embodiment, a safety needle assembly includes a cannula, a hub on an end of the cannula, a housing that is slidable with respect to the cannula and a tether that prevents the housing from extending past the distal end of the cannula. In one embodiment, a spring washer prevents the housing from being moved in a proximal direction after the housing has covered the distal end of the needle.

### FIELD OF THE INVENTION

Embodiments of the present invention relate to a needle safety assembly and more particularly to a safety shield assembly that may be used in conjunction with a syringe assembly, a hypodermic needle, a needle assembly, a needle assembly with a needle holder, an intravenous catheter assembly, a blood collection needle, a blood collection set, an intravenous infusion set or other fluid handing devices or assemblies that contain piercing elements. Embodiments of the present invention generally relate to needle assemblies. Specific embodiments of the invention more particularly relate to safety needles that include a housing that is slidable along the cannula to cover and lock over the sharp distal needle point after the needle has been used.

### BACKGROUND

Hypodermic syringes are widely used in the medical arts for administering medicaments and for drawing body fluid samples. Generally, hypodermic syringes include a fixedly or removably attached metal needle that has a sharpened distal point for penetrating vial stoppers or a patient's skin. Hypodermic syringes and needles have been used for many years with few problems reported, taking into consideration the vast numbers of needles used. More recently, with the recognition of viral diseases that are transmitted by body fluids and greater sensitivity of the need to protect health care workers from inadvertent contact with previously used needles (commonly referred to as “sharps”), syringes and needles that include provisions to prevent needle stick injuries have been developed.

Provisions intended to protect health care workers from accidental needle sticks include a variety of sharps collector systems that are widely used in health care facilities. Other developments include needle attachments that may be readily broken off by practitioners after the syringe has completed its intended use. A variety of shielding mechanisms have been developed which are intended to shield the needle or sharp after it has been used, thus reducing the risk of an accidental needle stick, such as retracting needle syringes. Nevertheless, many of these provisions go unused, or are under utilized, either because the system is somewhat inconvenient for the user, or because of excessive expense.

Accordingly, a need exists for a safety needle that can protect against accidental needle stick injuries that is both inexpensive to manufacture and is convenient to use.

### SUMMARY

Embodiments of the invention pertain to a safety needle assembly. In one embodiment, a safety needle assembly comprises a cannula having a proximal end and a distal end with a point; a hub disposed on the proximal end of the cannula; a housing partially surrounding a portion of the cannula, the housing having a distal end; an extensible tether having a first end secured to the hub and a second end secured to the housing; and a spring washer associated with the distal end of the housing, the spring washer having a spring force and a substantially central aperture sized to permit the cannula to slide therethrough and to allow sliding movement of the housing along the length of the cannula, the spring washer preventing the housing from being moved in a proximal direction along the length of the cannula when the housing has surrounded the needle point. In one embodiment, the spring washer is mounted on the distal end of the housing. The spring washer is a Belleville washer. According to certain embodiments, the Belleville washer includes radial slots extending from the substantially central aperture.

In one or more embodiments, the extensible tether comprises a rolled sheet of material and the first end of the tether is an inner end and the second end of the tether is an outer end. The rolled sheet of material may be rolled into a spiral configuration. According to certain embodiments, the outer end of the rolled material is attached to an inner surface of the housing. In certain embodiments, the extensible tether has an extension length that permits the cannula point to pass through the aperture while preventing a proximal end of the housing from extending beyond the distal end of the cannula.

The extensible tether may comprise a plurality of substantially concentric shells. In one or more embodiments, the tether extends to form a substantially cone-shaped enclosure around the cannula as housing is moved distally along the length of the cannula. In certain embodiments, the tether is a spiral wound sheet of material that is axially extensible and envelopes the cannula as the housing is moved distally along the length of the cannula. According to one or more embodiments, the assembly cannula is a spinal or epidural cannula

### BRIEF DESCRIPTION OF THE DRAWINGS

FIG. 1 is partial cross-sectional view of a safety needle assembly according to one embodiment;

FIG. 2 is partial a cross-sectional view of the safety needle assembly of FIG. 1 showing the tether partially extended;

FIG. 3 is a plan view of a spring washer device according to one embodiment;

FIG. 4 is a perspective view of a first embodiment tether;

FIG. 5 is a partial cross-sectional view of the assembly shown in FIG. 1 showing the sharp point of the needle enclosed by a housing;

FIG. 6 is a partial cross-sectional view of an over-the-needle catheter assembled with the safety needle shown in FIG. 1;

FIG. 7 is a perspective view of another embodiment of a tether;

FIG. 8 shows a partial cross-sectional view of another embodiment of a safety needle assembly a member for preventing the housing from sliding in a proximal direction after the housing surround the needle point; and

FIG. 9 shows the needle assembly of FIG. 7 after deployment.

### DETAILED DESCRIPTION

Before describing several exemplary embodiments of the invention, it is to be understood that the invention is not limited to the details of construction or process steps set forth in the following description. The invention is capable of other embodiments and of being practiced or being carried out in various ways. In this disclosure, a convention is followed wherein the distal end of the device is the end closest to a patient and the proximal end of the device is the end away from the patient and closest to a practitioner.

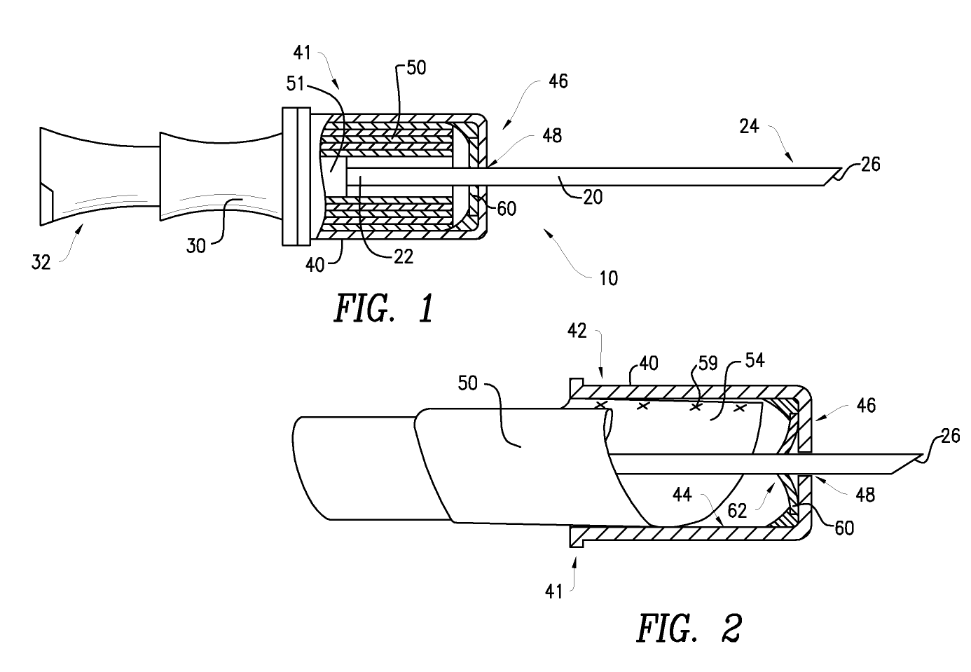

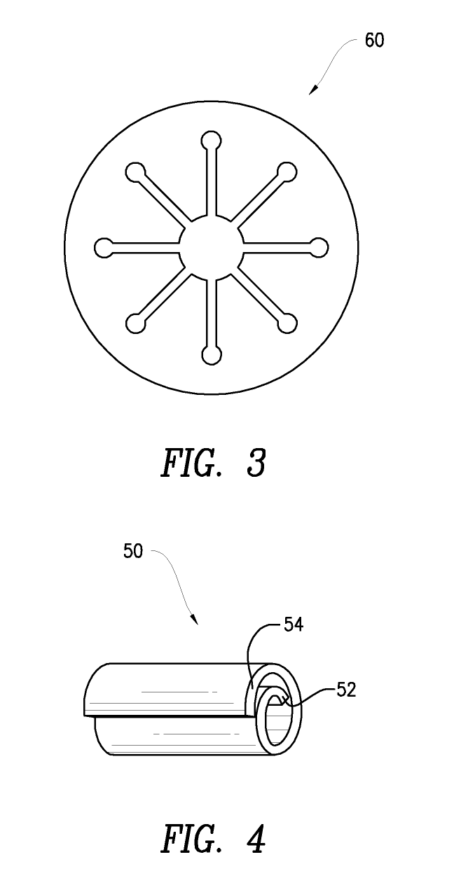

Referring generally to FIGS. 1-5, a safety needle assembly 10 according to a first embodiment of the present invention is shown. The safety needle assembly 10 includes a cannula 20, a hub 30 and a housing 40. The cannula 20 may be a long needle, adapted for spinal or epidural procedures. For example, the needle cannula 20 may be a Quincke type spinal needle having a gauge in the range from about 18 to 27 and in lengths from about 1″ to 7″, or the cannula may be a Whitacre type spinal having a gauge from about 22 to 27 lengths from about 3½″ to 5″. Of course, other types of spinal needles could be used. The cannula may also be an epidural needle having a gauge of about 17 to 22 and 2 to 3 inches in length. These needle cannulas are exemplary only, and it will be understood that the present invention is not limited to a particular needle gauge, type or length.

The cannula 20 has a proximal end 22 fixed to the hub 30, and a distal end 24 that terminates at a sharp distal point 26. It will be appreciated that the distal end 24 does not necessarily have to terminate at a sharp distal point, and the distal end 24 may have a blunt tip. The hub 30 may be any suitable device known in the art, and may have flanges 32 adapted to mate with a locking luer collar, for example. Other types of hubs 30 are possible, such as providing a screw cap adapted to screw onto a corresponding male member of a medical device, which may be a syringe, catheter, etc. Alternatively, the hub 30 may be the base of a syringe to which the needle assembly 10 is permanently attached. Any method known in the art may be used to fix the hub 30 onto the proximal end 22 of the cannula 20, such as adhesives, welding, mechanical engagement or the like.

The housing 40 provides an enclosure or shell that surrounds and covers a portion of the cannula 20. The housing 40 may be, for example, substantially cylindrical in shape, and made of plastic, metal or any other sufficiently tough material that can resist puncturing by the point 26 of the cannula 20. The housing 40 has an outer surface 42, which serves as a grip for the practitioner, and an inner surface 44. The outer surface 42 may be knurled or otherwise be provided with a roughened surface to provide a gripping surface for the user. The housing 40 has an end face 46 with an aperture 48. The cannula 20 passes through the aperture 48. As a result, the housing 40 may slide proximally and distally along the cannula 20. Generally, however, the housing 40 is kept in a retracted position, so that the most proximal end 41 of the housing 40 abuts the hub 30. Although the outer surface 42 of the housing 40 is preferentially solid, it will be appreciated that the outer surface 42 could also have openings, such as holes, slits, or the like to reduce on material costs. For purposes of this disclosure, such a configuration is still considered to cover that portion of the cannula 20 over which such a housing 40 is disposed.

The housing 40 provides a safety shield that covers the sharp point 26 after the safety needle 10 has been used. After use, the practitioner grabs the outer surface 42 and distally slides the housing 40 from the hub 30 to the sharp point 26. After the sharp point 26 has passed through second aperture 62, the point 26 will be safely enclosed within the housing 40. Once enclosed, the practitioner and others are shielded from any accidental stabs from the sharp point 26. However, this shielding function would be frustrated if the proximal end 41 of the housing 40 were capable of moving distally beyond the distal end 24 of the cannula, i.e., past the sharp point 26. Hence, the safety needle 10 further comprises an extensible tether 50, which connects the housing 40 to the hub 30. As the housing 40 moves distally along the cannula 20, the tether 50 extends up to, but not beyond, a predetermined length. The tether 50 thus provides an extensible length beyond which the housing 40 may not distally extend, as further explained below.

The tether 50 may be, for example, a rolled material, such as rolled plastic, metal or fabric in sheet form. With this configuration, the tether 50 has an inner end 52 and an outer end 54. In the embodiments shown, the outer end 54 is fixed to the inner surface 44 of the housing 40, which may be accomplished by any suitable fastening mechanism 59. Suitable fastening mechanisms may include rivets, heat sealing, adhesives and the like. The inner end 52 of the tether 50 is fixed to the hub 30, by fastening mechanism 51. Similar to fastening mechanism 46 for the outer end 54, the inner end 52 may be fastened by rivets, heat sealing, welding, adhesives or any other suitable fastening mechanism. In the embodiment shown, as the rolled tether 50 distally extends along the cannula 20, the concentric rolls of the tether 50 slide past each other to cover or completely envelope the cannula 20, further shielding the practitioner from any biological materials present on the outside surface of the cannula 20. The tether 50 deploys in the form of a cone-shaped enclosure around the cannula 20. The inner roll 52 of the tether 50 remains fixed to the hub 30, while the outer roll 54, fixed to the inner surface 44, moves with the housing 40 towards the distal end 24 of the cannula. The tether 50 is configured, as by the number of rolls, to provide a fully-extended length that prevents the proximal end 41 of the housing 40 from distally extending beyond the sharp point 26 of the cannula 20, while being sufficiently long as to permit the sharp point 26 to pass fully through the second aperture 62 of the end face 46.

Exposure of the sharp point 26 by passing beyond the proximal end 41 of the housing 40 is prevented by the tether 50. However, it is also necessary to prevent exposure of the sharp point 26 by reinsertion through the aperture 48. In FIGS. 1-5, a spring washer 60 is thus provided, which prevents the sharp point 26 of the cannula 20 from passing back through the aperture 48. It will be appreciated that the spring washer 60 may be mounted to the distal end of the housing, or it may be an integral part of the housing. For example, the spring washer may be integrally molded into the housing. The spring washer is a flexible disc having an opening 62 therethrough, the diameter of the opening which increases as the disc is flexed.

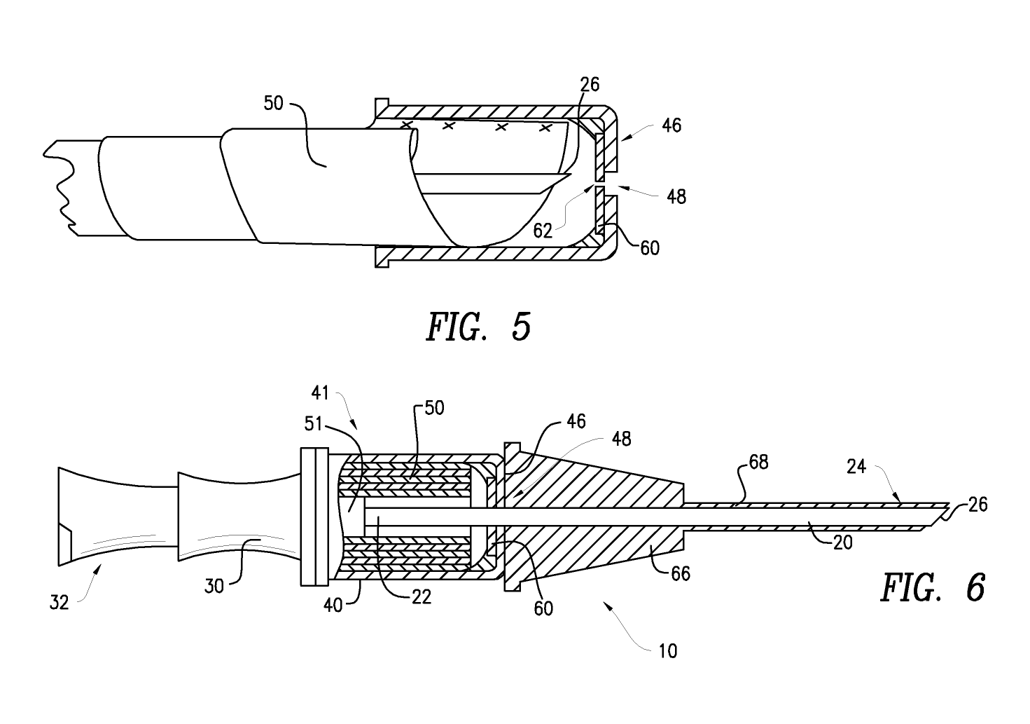

The spring washer 60 may be, for example, a Belleville washer 60, disposed on the inner surface 44 of the end face 46. Initially, when the cannula 20 passes through the aperture 48, the Belleville washer 60 extends proximally to provide a second aperture 62 that is sufficiently wide to accept the cannula 20; in this condition, the Belleville washer 60 is in a biased state due to the width of the cannula 20. As the housing 40, and hence the Belleville washer 60, slides distally along the cannula 20, the direction of movement of the Belleville washer 60 corresponds to the proximal extension of the Belleville washer 60, and hence distal movement is facilitated by the Belleville washer 60. However, proximal movement of the housing 40 may be discouraged by the Belleville washer 60, as such movement tends to cause the Belleville washer 60 to lock onto the cannula 20. After the cannula 20 has passed completely through the apertures 48 and 62, the Belleville washer 60 may fully relax into an unbiased state, thus reducing the width of the second aperture 62, as shown in FIG. 5. If the sharp point 26 attempts to pass back through the aperture 48, the Belleville washer 60, buttressed by the face 46 of the housing 40, blocks reentry of the cannula 20 through the aperture 48. Hence, the sharp point 26 remains safely disposed within the housing 40, prevented by the tether 50 and the aperture restriction device 60 from exiting the confines of the housing 40.

The safety needle assembly 10 can be used with various medical devices. FIG. 6 shows the safety needle assembly 10 shown together with an over-the-needle catheter including a hub 66 and catheter portion 68. Housing 40 enclosing tether 50 of safety needle assembly 10 is shown abutting catheter hub 66 in an assembled configuration. As will be appreciated by the skilled artisan, after the needle and catheter have been inserted into a patient, the needle 20 is withdrawn from the patient and through the catheter 68, and the tether 50 will extend until the point 26 of the needle moves past the aperture 48 and spring washer 60 to capture the point within housing 40 as described above. The needle assembly 10 can then be safely discarded and an appropriate hub or tubing can then be connected to the catheter hub 66.

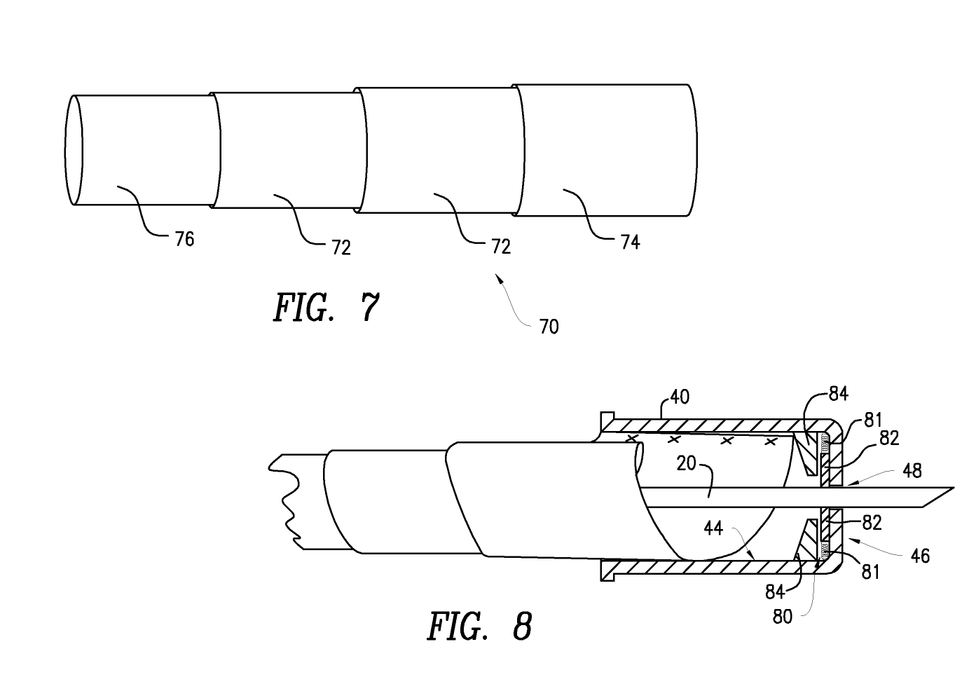

Other configurations for the tether 50 are possible. For example, as shown in FIG. 7, a tether 70 may be provided in place of the tether 50. The tether 70 may include a plurality of substantially concentric shells 72. The outermost shell 74 may be affixed to the inner surface 44 of the housing, and the innermost shell 76 may be affixed to the hub 30. The shells 72, 74, 76 are designed to telescopically slide with respect to each other, but not to extend past each other, and the total extension length of the tether 70 is long enough to permit the sharp point 26 to pass within the housing 40, while preventing the proximal end 41 of the housing 40 from extending beyond the sharp point 26. As with the tether 50, the tether 70 has the advantage of fully covering the cannula 20 when the housing 40 is maximally extended to cover and shield the sharp point 26.

Alternatively, other means for preventing the housing from extending beyond a distal end of the cannula may be provided. For example, the tether 50 may be replaced by a line of material. This line may be elastic or non-elastic, and may be disposed within the housing 40 to feed out as the housing 40 advances distally. One end of the line may be attached to the housing 40, such as to the inner surface 44, and the other end may be attached to the hub 30.

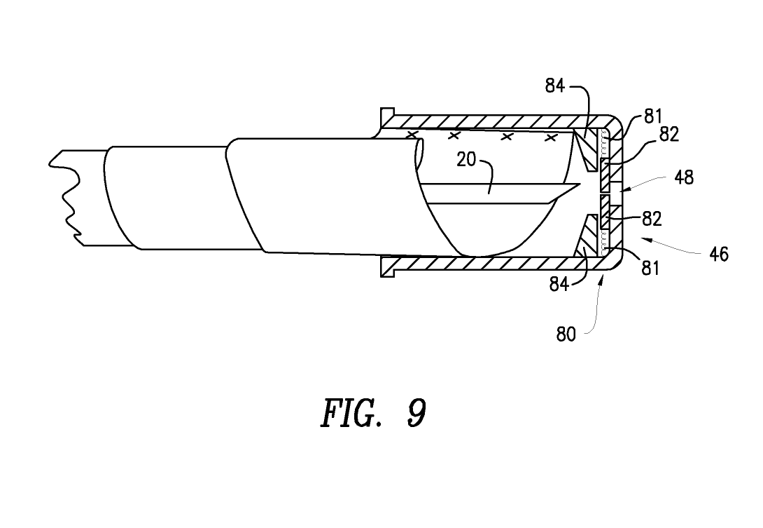

The spring washer 60 may be replaced by other devices for preventing proximal movement of the housing after the covering housing covers the distal end of the cannula. For example, as shown in FIGS. 8 and 9, an aperture restriction device 80 includes one or more shutters 82 elastically biased against the inner surface 44 of the housing 40. The shutters 82 are slidably disposed between the inner surface 44 of the face 46 and respective guide walls 84. Springs 81 bias the shutters 82 to move towards the aperture 48 to cover the aperture 48. As shown in FIG. 7, the shutters 82 initially press against the cannula 20; however, after the cannula 20 has fully retracted into the housing 40, the shutters 82 are free to move together, thus blocking the cannula 20 from reentering the aperture 48, as shown in FIG. 8. Although multiple shutters 82 are shown, it should be clear that a single shutter 82 may be used to obstruct the aperture 48.

While the foregoing is directed to embodiments of the present invention, other and further embodiments of the invention may be devised without departing from the basic scope thereof, and the scope thereof is determined by the claims that follow.

What is claimed is:

1. A safety needle assembly comprising:
a cannula having a proximal end and a distal end;
a hub disposed on the proximal end of the cannula;
a housing partially surrounding a portion of the cannula, the housing having a distal end;
an extensible tether having a first end secured to the hub and a second end secured to the housing; and
a spring washer associated with the distal end of the housing, the spring washer having a spring force and a substantially central aperture sized to permit the cannula to slide therethrough and to allow sliding movement of the housing along the length of the cannula, the spring washer preventing the housing from being moved in a proximal direction along the length of the cannula after the housing has surrounded the distal end of the needle.

2. The safety needle assembly of claim 1 wherein the spring washer is mounted on the distal end of the housing.

3. The safety needle assembly of claim 2 wherein the spring washer is a Belleville washer.

4. The safety needle assembly of claim 3 wherein Belleville washer includes radial slots extending from the substantially central aperture.

5. The safety needle assembly of claim 1 wherein the extensible tether comprises a rolled sheet of material and the first end of the tether is an inner end and the second end of the tether is an outer end.

6. The safety needle assembly of claim 5 wherein the rolled sheet of material is rolled into a spiral configuration.

7. The safety needle assembly of claim 6 wherein the outer end of the rolled material is attached to an inner surface of the housing.

8. The safety needle assembly of claim 7 wherein the spring washer is a Belleville washer.

9. The safety needle assembly of claim 7 wherein the extensible tether has an extension length that permits the cannula distal end to pass through the aperture while preventing a proximal end of the housing from extending beyond the distal end of the cannula.

10. A catheter assembly including the needle safety assembly of claim 1.

11. The safety needle assembly of claim 1 wherein the extensible tether has an extension length that permits the distal end of the cannula to pass through the aperture while preventing a proximal end of the housing from distally extending beyond the distal end of the cannula.

12. The safety needle assembly of claim 1 wherein the extensible tether comprises a plurality of substantially concentric shells.

13. The safety needle assembly of claim 12 wherein the tether extends to form a substantially cone-shaped enclosure around the cannula as housing is moved distally along the length of the cannula.

14. The safety needle assembly of claim 1 wherein spring washer is molded into the housing.

15. A safety needle assembly comprising:
a cannula having a proximal end and a distal end;
a hub disposed on the proximal end of the cannula;
a housing partially surrounding a portion of the cannula, the housing having a distal end, the housing movable along the length of the cannula and secured to the assembly by a tether; and
a Belleville washer disposed at the distal end of the housing to prevent the housing from being moved in a proximal direction along the length of the cannula after the Belleville washer has been moved to the distal end of the cannula.

16. The safety needle assembly of claim 15 wherein the tether is a spiral wound sheet of material that is axially extensible and envelopes the cannula as the housing is moved distally along the length of the cannula.

17. The safety needle assembly of claim 16 wherein the Belleville washer includes a plurality of axial slots.

18. A safety needle assembly comprising:
a cannula having a proximal end secured to a hub and distal end;
a housing slidable with respect to the cannula and covering a portion of the cannula;
means for preventing the housing from extending beyond a distal end of the cannula; and
means for preventing proximal movement of the housing after the covering housing covers the distal end of the cannula.

19. The safety needle assembly of claim 18 wherein the means for preventing proximal movement of the housing comprises a spring washer.

20. The safety needle assembly of claim 19 wherein the spring washer is an axially slotted Belleville washer.
